(19) United States
(12) Patent Application Publication
(10) Pub. No.: US 2023/0115558 A1
(43) Pub. Date: Apr. 13, 2023

(54) STERILIZED ENDOSCOPE KIT, AND METHOD FOR MANUFACTURING STERILIZED ENDOSCOPE KIT

(71) Applicant: OLYMPUS MEDICAL SYSTEMS CORP., Tokyo (JP)

(72) Inventors: Masahiro ASHIZUKA, Tokyo (JP); Kester J. BATCHELOR, Mound, MN (US)

(73) Assignee: OLYMPUS MEDICAL SYSTEMS CORP., Tokyo (JP)

(21) Appl. No.: 17/960,358

(22) Filed: Oct. 5, 2022

Related U.S. Application Data
(60) Provisional application No. 63/253,736, filed on Oct. 8, 2021.

(51) Int. Cl. A61B 1/00; A61B 1/018

(52) U.S. Cl. CPC A61B 1/00144; A61B 1/018; A61M 25/002

(57) ABSTRACT

A sterilized endoscope kit, comprising a sterilized endoscope, a sterilized treatment instrument, a sterilized guide wire, and a packaging material. The sterilized endoscope includes a first lumen. The sterilized treatment instrument is inserted into the first lumen, the sterilized treatment instrument includes a second lumen. The sterilized guide wire is inserted into the second lumen. The sterilized endoscope, the sterilized treatment instrument, and the sterilized guide wire are arranged in the packaging material, and the packaging material provides a sterile environment for the sterilized endoscope, the sterilized treatment instrument, and the sterilized guide wire. A method for manufacturing a sterilized endoscope kit, comprising inserting a sterilized guide wire into a first lumen of a sterilized treatment instrument, inserting the sterilized treatment instrument into a second lumen of a sterilized endoscope, and placing the sterilized guide wire, the sterilized treatment instrument, and the sterilized endoscope into a packaging material.

### RELATED APPLICATION DATA

This application is based on and claims priority under 37 U.S.C. § 119 to U.S. Provisional Application No. 63/253,736 filed on Oct. 8, 2021, the entire contents of which are incorporated herein by reference.

### FIELD OF THE DISCLOSURE

The present disclosure relates to a sterilized endoscope kit including an endoscope and a treatment instrument, and a method for manufacturing the sterilized endoscope kit.

### BACKGROUND

Conventionally, treatment instruments used with endoscopes have been known. For example, Japanese Patent Application Laid-Open Publication No. 2004-275785 discloses a high frequency dissection instrument that is used in endoscopic sphincterotomy (EST). In EST, there is known a procedure of inserting a high frequency dissection instrument into a papillary opening portion and dissecting a papilla, thereafter, inserting a guide wire into a bile duct (or a pancreatic duct), extracting the high frequency dissection instrument after insertion of the guide wire, and guiding a basket, forceps or the like to the bile duct (or the pancreatic duct) along the guide wire.

As an endoscope that inserts a treatment instrument into a site to be treated, a configuration in which a raising base (forceps elevator) that makes a direction of a treatment portion variable is provided at a distal end portion of an insertion portion is well known, as disclosed in Japanese Patent Application Laid-Open Publication No. 2004-321822, for example.

### SUMMARY OF THE DISCLOSURE

A sterilized endoscope kit, comprising a sterilized endoscope, a sterilized treatment instrument, a sterilized guide wire, and a packaging material. The sterilized endoscope includes a first lumen. The sterilized treatment instrument is inserted into the first lumen, the sterilized treatment instrument includes a second lumen. The sterilized guide wire is inserted into the second lumen. The sterilized endoscope, the sterilized treatment instrument, and the sterilized guide wire are arranged in the packaging material, and the packaging material provides a sterile environment for the sterilized endoscope, the sterilized treatment instrument, and the sterilized guide wire.

A method for manufacturing a sterilized endoscope kit, comprising inserting a sterilized guide wire into a first lumen of a sterilized treatment instrument, inserting the sterilized treatment instrument into a second lumen of a sterilized endoscope, and placing the sterilized guide wire, the sterilized treatment instrument, and the sterilized endoscope into a packaging material.

### BRIEF DESCRIPTION OF THE DRAWINGS

FIG. 1 is a plan view showing a configuration of a sterilized endoscope kit of one aspect of the present disclosure.

FIG. 2 is a plan view showing a state before an endoscope, a treatment instrument, and a guide wire are accommodated in a sterilization pack.

FIG. 3 is a sectional view showing a distal end portion of an insertion portion in a state in which a tube body of a treatment instrument, and the guide wire inserted into the tube body are inserted into a treatment instrument insertion channel, and a treatment instrument raising base is raised.

FIG. 4 is a sectional view showing a state in which a cap is fitted onto the distal end portion of the insertion portion in which the tube body of the treatment instrument, and the guide wire inserted into the tube body are inserted into the treatment instrument insertion channel.

FIG. 5 is a perspective view partially showing a configuration in which a fixed block is integrally formed at a forceps plug provided at an operation portion of the endoscope.

FIG. 6 is a perspective view partially showing a configuration in which a fixed block is integrally formed at a guide wire insertion pipe sleeve of a papillotomy knife.

FIG. 7 is a perspective view showing a sterilized endoscope kit in which a packaging material is a blister pack.

FIG. 8 is a plan view showing the sterilized endoscope kit in which the packaging material is a blister pack.

### DETAILED DESCRIPTION OF THE PREFERRED EMBODIMENT

Hereinafter, a sterilized endoscope kit of the present disclosure is described. Note that in the following explanation, the drawings based on each embodiment are schematic, attention should be paid to the fact that the relationship between the thickness and the width of each part, ratios of the thicknesses of respective parts and the like are different from the actual relationship and ratios, and there may be parts where the relationships and ratios of mutual dimensions are different among the drawings.

Further, as an endoscope in the following explanation, a duodenal endoscope and a treatment instrument for endoscope such as a papillotomy knife that are used in endoscopic sphincterotomy (EST) are illustrated.

Note that the endoscope is not limited to the duodenal endoscope or the treatment instrument for endoscope such as a papillotomy knife, but the present disclosure can also be applied to so-called various flexible endoscopes in which insertion portions have flexibility to be inserted into digestive organs in an upper part or a lower part of a living body, so-called rigid endoscopes with rigid insertion portions used for surgery, and various treatment instruments for endoscope such as basket forceps, contrast agent cannulas, biopsy forceps, and local injection needles that are used with the various endoscopes.

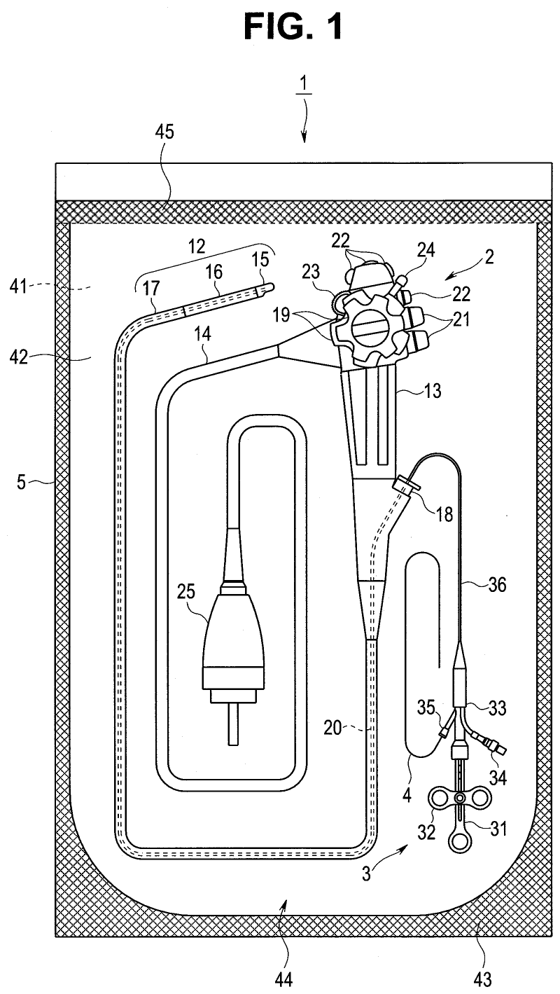

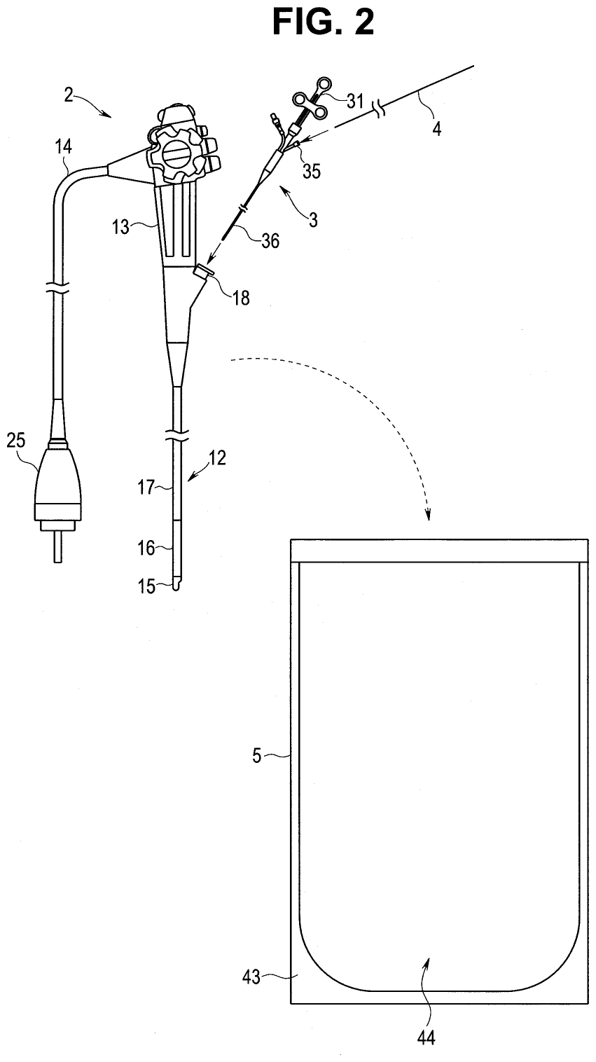

A sterilized endoscope kit 1 of a present embodiment includes a package unit in which an endoscope 2, a papillotomy knife 3 that is a treatment instrument for endoscope, and a guide wire 4 are put into a sterilization pack 5 that is a packaging material for sterilization, as shown in FIG. 1.

First, a schematic configuration of the endoscope 2 of the present embodiment is described. Note that in the present embodiment, as the endoscope 2, a mode that optically observes an inside of a subject, and has a treatment instrument raising base at a distal end portion is illustrated.

The endoscope 2 is mainly configured by including an insertion portion 12 that is an elongated member insertable into the inside of the subject, an operation portion 13 that is located at a proximal end of the insertion portion 12, and a universal cord 14 that is a composite cable extending from a side portion of the operation portion 13.

The insertion portion 12 is configured by a distal end portion 15 that is placed at a distal end, a bendable bending portion 16 that is placed at a proximal end side of the distal end portion 15, and a flexible tube portion 17 that has flexibility, is placed at a proximal end side of the bending portion 16 and is connected to a distal end side of the operation portion 13, being provided to be connected in series.

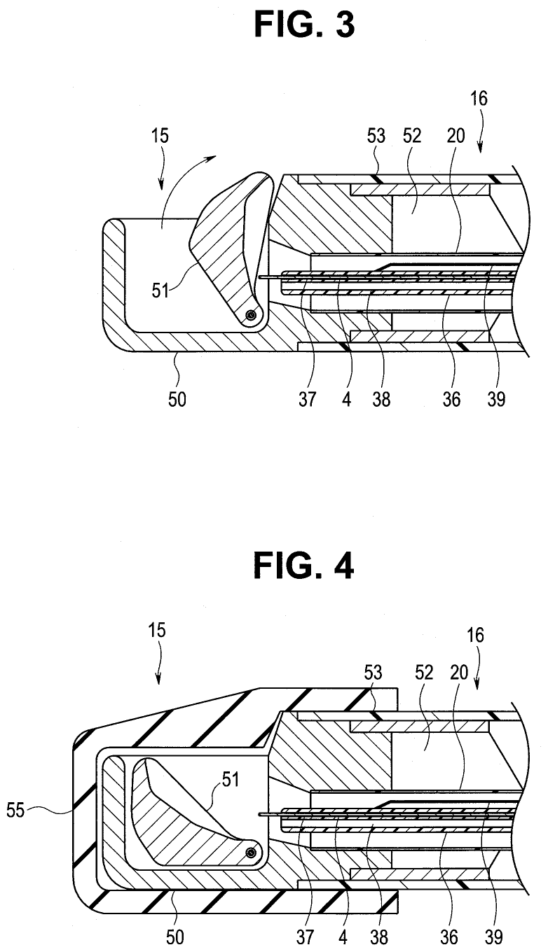

The operation portion 13 is provided with a forceps plug 18 that is a forceps pipe sleeve at a treatment instrument insertion port communicating with a treatment instrument insertion channel 20 of a conduit that is a lumen. The operation portion 13 is provided with an angle knob 19 for operating to bend the bending portion 16, buttons 21 such as air feeding and water feeding buttons for performing control of a delivery action of a fluid from a fluid delivery portion (not illustrated) provided in the distal end portion 15, switches 22 such as a release switch provided at the distal end portion 15, an operation lever 23 configured to operate to raise and lower a treatment instrument raising base 51 (see FIG. 3 and FIG. 4) provided at the distal end portion 15, and a fixing lever 24 configured to fix the angle knob 19. The forceps plug is also called a biopsy valve.

Note that the distal end portion 15 has a distal end rigid portion 50 configured to turnably hold the treatment instrument raising base 51, with the treatment instrument insertion channel 20 connected to the distal end rigid portion 50 (see FIG. 3 and FIG. 4). To the distal end rigid portion 50, a distal end of a bending piece 52 arranged in the bending portion 16 is fitted, and a distal end of a bending rubber 53 that covers an outer periphery of the bending portion 16 is fixedly attached by bobbin winding bonding or the like not illustrated.

An endoscope connector 25 that is connected to a video processor (not illustrated) including a light source apparatus is provided on a proximal end side of the universal cord 14. Illumination light emitted from a light source of the video processor is transmitted to a light guide bundle not illustrated that is inserted through the universal cord 14, the operation portion 13, and the insertion portion 12, and is emitted from an illumination window (not illustrated) provided at the distal end portion 15. Note that the endoscope 2 may have a configuration including a light source such as LEDs at the distal end portion 15 as an illumination device.

The distal end portion 15 is provided with an observation window (not illustrated), and contains an image sensor (not illustrated) that photoelectrically changes photographing light entering from the observation window. An image pickup cable extending from the image sensor is also inserted through the insertion portion 12, the operation portion 13, and the universal cord 14 to be placed to the endoscope connector 25.

Note that since the endoscope 2 here is a side-viewing endoscope including diagonal viewing, the observation window and the illumination window are provided at side portions of the distal end portion 15 of the insertion portion 12. Note that the endoscope 2 is preferably a disposable (single-use) endoscope that is discarded after use, but of course, may be a reuse product that is disinfected and sterilized, and reused.

Next, a schematic configuration of a papillotomy knife 3 that is a treatment instrument for endoscope is described. The papillotomy knife 3 has an operation portion 31 provided with a slider 32, a branch portion 33 connected to the operation portion 31, a liquid feeding pipe sleeve 34 branched by the branch portion 33, a guide wire insertion pipe sleeve 35, and a tube body 36 of an insertion portion that is a long member provided to extend from the branch portion 33.

The slider 32 of the operation portion 31 is provided with an electrode plug and a current application button not illustrated, and an end portion of a metal dissection wire 39 (see FIG. 3 and FIG. 4) that is inserted through an inside of the tube body 36 is connected to the slider 32. The dissection wire 39 is exposed on a distal end portion of the tube body 36 by a predetermined length, and is folded back to be inserted through the inside of the tube body 36.

In the dissection wire 39, a portion exposed on the distal end portion of the tube body becomes bow-shaped by an operation of the slider 32, and dissects living tissue, in particular, a papilla, by energization.

Note that the tube body 36 is a multi-lumen tube that is formed from a synthetic resin such as a fluororesin and has a wire insertion passage 37 that is a lumen allowing insertion of the guide wire 4, and a flow path 38 that is a lumen for feeding a liquid such as a contrast agent.

The guide wire insertion pipe sleeve 35 is provided with a silicon rubber (not illustrated) for watertightness, and allows insertion of the guide wire 4.

The guide wire 4 is, for example, a long wire made of a superelastic alloy such as a nickel-titanium alloy having a fluorine coating on a core metal. The guide wire 4 is inserted from the guide wire insertion pipe sleeve 35 of the papillotomy knife 3, and is inserted through the wire insertion passage 37 of the tube body 36.

Next, a schematic configuration of the sterilization pack 5 that is a packaging material for sterilization is described.

The sterilization pack 5 has a sterile sheet 41 such as sterile paper specially made, and a transparent sheet 42 such as a clean PE film, and has a heat seal 43 applied to three edge sides by sealing such as thermal welding so as to be in a bag shape. The sterile sheet 41 seals around one or more of the sterilized guide wire 4, the sterilized treatment instrument, and the sterilized endoscope 2.

After the endoscope 2, the papillotomy knife 3 and the guide wire 4 are accommodated in a space 44 in the pack from an opening, a heat seal 45 is applied to the sterilization pack 5 by sealing such as thermal welding.

At this time, the guide wire 4 is inserted from the guide wire insertion pipe sleeve 35 of the papillotomy knife 3, and is disposed in the wire insertion passage 37 in advance. The tube body 36 of the papillotomy knife 3 is also inserted from the forceps plug 18 of the endoscope 2 and is disposed in the treatment instrument insertion channel 20 in advance.

In other words, the sterilization pack 5 has one remaining open side sealed after the endoscope unit in a state in which the endoscope 2, the papillotomy knife 3, and the guide wire 4 are incorporated is put into the sterilization pack 5, and is brought into a hermetically sealed state in which all four sides are subjected to the heat seals 43 and 45, from the three-way seal sterilization bag state. Note that the sterile sheet 41 has a structure that prevents inflow of bacteria and passes gas such as ethylene oxide gas for sterilization, or high-temperature and high-pressure steam of an autoclave or the like.

In this way, the tube body 36 of the papillotomy knife 3 is incorporated into the endoscope 2, and the guide wire 4 is incorporated into the tube body 36, in advance, the endoscope 2, the tube body 36 and the guide wire 4 are put into the sterilization pack 5, and are subjected to sterilization treatment by ethylene oxide gas sterilization or the like, whereby the sterilized endoscope kit 1 is provided.

In other words, the sterilized endoscope kit 1 has a configuration in which the guide wire 4 and the tube body 36 of the papillotomy knife 3 are inserted into the treatment instrument insertion channel 20 of the endoscope 2 in advance and are subjected to sterilization treatment.

More specifically, as a method for manufacturing the sterilized endoscope kit 1 of the present embodiment, a worker on a product manufacture side, or a health care worker such as a nurse inserts the guide wire 4 that is a wire into the wire insertion passage 37 that is a first lumen of the papillotomy knife 3 that is a treatment instrument, inserts the guide wire 4 and the papillotomy knife 3 into the treatment instrument insertion channel 20 that is a second lumen of the endoscope 2, places the guide wire 4, the papillotomy knife 3 and the endoscope 2 in an inside of the sterilization pack 5 that is a packaging material, and sterilizes the inside of the sterilization pack 5.

Thereby, the sterilized endoscope kit 1 can save a trouble of inserting the guide wire 4 and the tube body 36 of the papillotomy knife 3 that is the treatment instrument into the treatment instrument insertion channel 20 of the endoscope 2 later, and a doctor who is a user can perform endoscopy and treatment quickly.

In other words, since in the sterilized endoscope kit 1, the guide wire 4 and the tube body 36 of the papillotomy knife 3 are inserted into the treatment instrument insertion channel 20 of the endoscope 2 in advance, a trouble of preparation work can be saved from a time when the sterilization pack 5 is unsealed.

Note that in the sterilized endoscope kit 1, the treatment instrument raising base 51 is brought into a raised state, as shown in FIG. 3, and thereby the guide wire 4 abuts on the treatment instrument raising base 51 to be restricted from moving and can be prevented from being drawn out from the distal end portion 15 of the insertion portion 12. The treatment instrument raising base 51 restricts movement of the sterilized guide wire 4 and the sterilized treatment instrument by raising the treatment instrument raising base 51.

Accordingly, the sterilization pack 5 can be prevented from being torn by the guide wire 4. In this way, the treatment instrument raising base 51 configures a holding portion that restricts movement of the guide wire 4 to a distal end side.

In the sterilized endoscope kit 1, the guide wire 4 may be prevented from popping out from the distal end portion 15 by fitting a cap 55 that is a lid body onto the distal end portion 15 of the insertion portion 12 as one of packaging materials as shown in FIG. 4. Thereby, the guide wire 4 abuts on the cap 55 to be restricted from moving, and is restrained from being drawn out from the distal end portion 15 of the insertion portion 12, so that the sterilization pack 5 can be prevented from being torn by the guide wire 4. In this way, the cap 55 that is a packaging material configures a holding portion that restricts movement of the guide wire 4 to the distal end side. The cap 55 is attached to a distal end of the sterilized endoscope 2, the cap 55 preventing one or more of the sterilized guide wire 4 and the sterilized treatment instrument from protruding outside of a lumen of the sterilized endoscope 2. The cap 55 may allow the passage of gas such as ETO or other sterilizing gases.

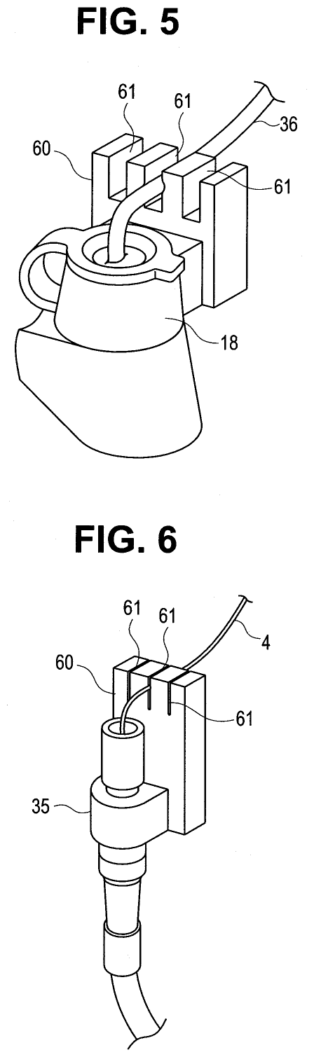

As show in FIG. 5, a fixed block 60 that is a holding portion configured to restrict movement of the tube body 36 of the papillotomy knife 3 may be integrally formed at the forceps plug 18 of the operation portion 13. The fixed block 60 is located at an operation portion 13 integrally formed at a forceps plug communicating with a lumen of the treatment instrument.

The fixed block 60 is formed of an elastic member of a rubber or the like in which a plurality of, three in this case, fixed grooves 61 are formed. The tube body 36 of the papillotomy knife 3 is held so as not to move by being inserted into any of the fixed grooves 61 of the fixed block 60.

Note that the fixed block 60 may be provided at the guide wire insertion pipe sleeve 35 of the papillotomy knife 3 as shown in FIG. 6. Here, the guide wire 4 is also held so as not to move by being inserted into any of the fixed grooves 61 of the fixed block 60.

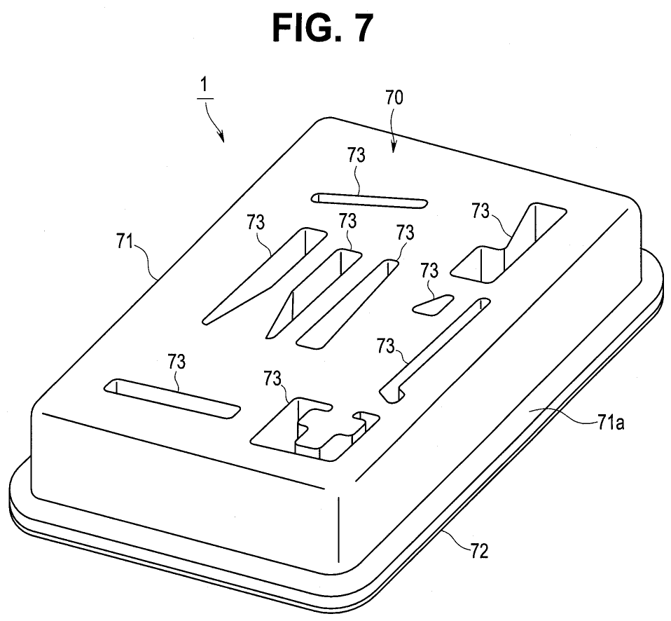

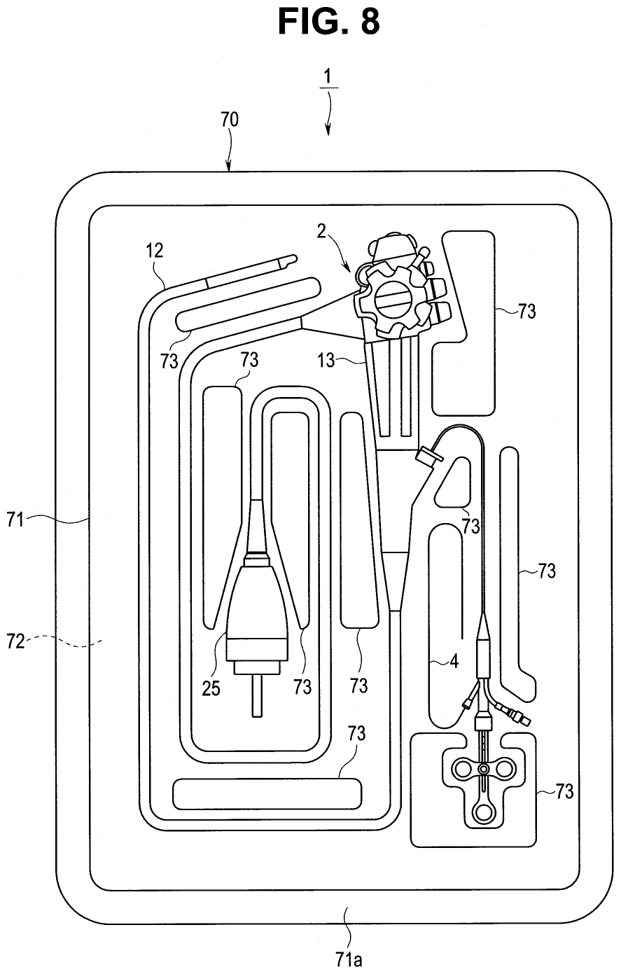

In the sterilized endoscope kit 1, a blister pack 70 that is a type of packaging material may be used instead of the sterilization pack 5 that is a packaging material for sterilization, as shown in FIG. 7 and FIG. 8.

The blister pack 70 has a substantially hat-shaped blister body 71 formed of transparent plastic such as PET (polyethylene terephthalate), or PVC (polyvinyl chloride), and backing paper 72 that is bonded to an edge portion 71a on four sides of the blister body 71 by sealing such as thermal welding. The packaging material includes a sterile sheet that is sealed around one or more of the sterilized guide wire, the sterilized treatment instrument, and the sterilized endoscope.

In the blister body 71, accommodation positions of the endoscope 2, the papillotomy knife 3 and the guide wire 4 are positioned, and a plurality of holding protruded portions 73 that restrict movement are formed by vacuum foam or the like. The blister pack 70 includes a holding portion including a first surface having a protrusion 73, and wherein the holding portion holds one of the sterilized guide wire, the sterilized treatment instrument, and the sterilized endoscope 2 stationary.

As the backing paper 72, sterile paper specially made or the like is also used here, and the backing paper 72 has a structure that prevents inflow of bacteria and passes gas such as ethylene oxide gas for sterilization or high-temperature and high-pressure steam of an autoclave or the like.

In this way, in the sterilized endoscope kit 1, it is possible to restrain the endoscope 2, the papillotomy knife 3, and the guide wire 4 from contacting each other and being damaged, by using the packaging material of the blister pack 70 including the plurality of holding protruded portions 73 that hold the endoscope 2, the papillotomy knife 3, and the guide wire 4 so that the endoscope 2, the papillotomy knife 3, and the guide wire 4 do not move. Since the endoscope 2, the papillotomy knife 3, and the guide wire 4 do not overlap, it is possible to stabilize sterilization processability.

The sterilized endoscope kit 1 described above has an advantage that the trouble of inserting the tube body 36 of the papillotomy knife 3 into the treatment instrument insertion channel 20 of the endoscope 2 and inserting the guide wire 4 into the tube body 36 after being unsealed can be saved, because the papillotomy knife 3 that is the treatment instrument and the guide wire 4 are in the state of being incorporated into the endoscope 2 and accommodated in the packaging material such as the sterilization pack 5, or the blister pack 70, and are sterilized.

The present disclosure is not limited to the aforementioned embodiment, and can be changed appropriately within the range without departing from the gist or the idea of the disclosure that can be read from the claims, the entire description, and the drawings.

(Example 1) A sterilized endoscope kit, comprising:

a first long member;

a second long member including a first lumen that allows insertion of the first long member, with the first long member being inserted into the first lumen in advance;

an endoscope including a second lumen that allows insertion of the second long member, with the second long member being inserted into the second lumen in advance; and

a packaging material configured to package the first long member, the second long member, and the endoscope.

(Example 2) The sterilized endoscope kit according to Example 1,

wherein the first long member is a guide wire.

(Example 3) The sterilized endoscope kit according to Example 2,

wherein the second long member is a member of a treatment instrument for endoscope.

(Example 4) The sterilized endoscope kit according to Example 1,

wherein the packaging material includes a holding portion configured to restrict movement of at least the endoscope.

(Example 5) The sterilized endoscope kit according to Example 4,

wherein the packaging material includes the holding portion configured to restrict movement of the first long member or the second long member.

(Example 6) The sterilized endoscope kit according to Example 1,

wherein the endoscope includes a second holding portion configured to restrict movement of the first long member or the second long member.

(Example 7) The sterilized endoscope kit according to Example 6,

wherein the second holding portion is provided at a distal end portion of an insertion portion or an operation portion of the endoscope.

(Example 8) The sterilized endoscope kit according to Example 7,

wherein the second holding portion provided at the operation portion is integrally formed at a forceps plug configured to close a proximal end portion of the second lumen.

(Example 9) The sterilized endoscope kit according to Example 7,

wherein the second holding portion is a treatment instrument raising base provided at the distal end portion, and restricts movement of the first long member by a state in which the treatment instrument raising base is raised.

(Example 10) The sterilized endoscope kit according to Example 1,

wherein the second long member includes a third holding portion configured to restrict movement of the first long member.

(Example 11) The sterilized endoscope kit according to Example 10,

wherein the third holding portion is provided at the second long member.

(Example 12) A method for manufacturing a sterilized endoscope kit, comprising:

inserting a first long member into a first lumen of a second long member;

inserting the first long member and the second long member into a second lumen of an endoscope;

placing the first long member, the second long member, and the endoscope in an inside of a packaging material; and

sterilizing the inside of the packaging material.

(Example 13) A sterilized endoscope kit, comprising:
an assembled endoscope; and
a packaging material,

wherein the assembled endoscope includes:
an endoscope,
a treatment instrument, and
a guide wire,

wherein the assembled endoscope is sterilized,

wherein the sterilized assembled endoscope is arranged in the packaging material, and

wherein the packaging material provides a sterile environment for the sterilized assembled endoscope.

(Example 14) A method for manufacturing a sterilized endoscope kit, comprising:
assembling an endoscope;
inserting the assembled endoscope into a packaging material;
sterilizing an inside of the packaging material to form a sterile environment; and
sealing the packaging material to seal the assembled endoscope in the sterile environment of the packaging material,

wherein the assembled endoscope includes:
a sterilized endoscope,
a sterilized treatment instrument, and
a sterilized guide wire
   (Example 15) The method for manufacturing the sterilized endoscope kit according to Example 14, wherein assembling the endoscope includes:
inserting the sterilized guide wire into a first lumen of the sterilized treatment instrument and
inserting the sterilized treatment instrument into a second lumen of the sterilized endoscope.

What is claimed is:

1. A sterilized endoscope kit, comprising:
a sterilized endoscope including a first lumen;
a sterilized treatment instrument inserted into the first lumen, the sterilized treatment instrument including a second lumen;
a sterilized guide wire inserted into the second lumen; and
a packaging material,
wherein the sterilized endoscope, the sterilized treatment instrument, and the sterilized guide wire are arranged in the packaging material, and
wherein the packaging material provides a sterile environment for the sterilized endoscope, the sterilized treatment instrument, and the sterilized guide wire.

2. The sterilized endoscope kit according to claim 1, wherein, during an insertion operation, the sterilized guide wire is configured to guide the sterilized treatment instrument along the sterilized guide wire, and wherein the sterilized treatment instrument is one of a forceps, a basket forceps, a contrast agent cannula, a biopsy forceps and a local injection needle.

3. The sterilized endoscope kit according to claim 1, wherein the packaging material restricts a movement of one or more of the sterilized guide wire, the sterilized treatment instrument, and the sterilized endoscope.

4. The sterilized endoscope kit according to claim 3, wherein the packaging material includes a sterile sheet that is sealed around one or more of the sterilized guide wire, the sterilized treatment instrument, and the sterilized endoscope.

5. The sterilized endoscope kit according to claim 3, wherein the packaging material includes a holding portion, wherein the holding portion includes a first surface having a protrusion, and wherein the holding portion holds one of the sterilized guide wire, the sterilized treatment instrument, and the sterilized endoscope stationary.

6. The sterilized endoscope kit according to claim 5, wherein the holding portion is a first holding portion, and
wherein the sterilized endoscope includes a second holding portion that restricts the movement of one or more of the sterilized guide wire and the sterilized treatment instrument.

7. The sterilized endoscope kit according to claim 6, wherein the sterilized treatment instrument includes a third holding portion that restricts the movement of the sterilized guide wire.

8. The sterilized endoscope kit according to claim 1, wherein the sterilized endoscope includes a holding portion that restricts a movement of one or more of the sterilized guide wire and the sterilized treatment instrument.

9. The sterilized endoscope kit according to claim 8, wherein the holding portion is located at a distal end portion of an insertion portion of the sterilized endoscope.

10. The sterilized endoscope kit according to claim 1, further comprising:
a cap attached to a distal end of the sterilized endoscope, the cap preventing one or more of the sterilized guide wire and the sterilized treatment instrument from protruding outside of the first lumen of the sterilized endoscope.

11. The sterilized endoscope kit according to claim 9, wherein the holding portion is a treatment instrument raising base, and wherein the treatment instrument raising base restricts movement of the sterilized guide wire and the sterilized treatment instrument by raising the treatment instrument raising base.

12. The sterilized endoscope kit according to claim 8, wherein the holding portion is located at an operation portion integrally formed at a forceps plug communicating with the second lumen.

13. The sterilized endoscope kit according to claim 12, wherein the holding portion includes a groove configured to hold the sterilized treatment instrument.

14. The sterilized endoscope kit according to claim 1, wherein the sterilized treatment instrument includes a groove configured to restrict a movement of the sterilized guide wire.

15. The sterilized endoscope kit according to claim 1, wherein the packaging material prevents inflow of bacteria and passes a gas.

16. A method for manufacturing a sterilized endoscope kit, comprising:
inserting a sterilized guide wire into a first lumen of a sterilized treatment instrument;
inserting the sterilized treatment instrument into a second lumen of a sterilized endoscope; and
placing the sterilized guide wire, the sterilized treatment instrument, and the sterilized endoscope into a packaging material.

17. The method for manufacturing the sterilized endoscope kit according to claim 16, further comprising:
sterilizing an inside of the packaging material.

18. The method for manufacturing the sterilized endoscope kit according to claim 16, further comprising:
fixing a placement of the sterilized guide wire relative to the sterilized treatment instrument.

19. The method for manufacturing the sterilized endoscope kit according to claim 18, further comprising:
fixing a placement of the sterilized treatment instrument relative to the sterilized endoscope.

20. The method for manufacturing the sterilized endoscope kit according to claim 19, further comprising:
fixing a placement of the sterilized endoscope relative to the packaging material.
